(19) United States
(12) Patent Application Publication
(10) Pub. No.: US 2013/0231520 A1
(43) Pub. Date: Sep. 5, 2013

(54) IMAG1 EYES MAGANETICS

(71) Applicants: GABE CHERIAN, SUN VALLEY, ID (US); DEREK SAKATA, SALT LAKE CITY, UT (US)

(72) Inventors: GABE CHERIAN, SUN VALLEY, ID (US); DEREK SAKATA, SALT LAKE CITY, UT (US)

(21) Appl. No.: 13/772,348

(22) Filed: Feb. 21, 2013

Related U.S. Application Data
(60) Provisional application No. 61/601,142, filed on Feb. 21, 2012.

(51) Int. Cl. A61N 2/00

(52) U.S. Cl. CPC A61N 2/004
USPC 600/12

(57) ABSTRACT

The invention provides solutions to the problem connected with detached eye retinas and to methods that can promote the healing of the repaired retina. Instead of injecting gas bubbles or silicone oil into the eyeball, the invention proposes to inject magnetic particles inside the eye and use external magnets to urge the magnetic particles to move against the repaired area of the retina, thus forcing the retina against the eyeball walls, thus promoting the healing. Optional eye movement sensors can optimize the distribution of the magnetic particles, to optimize the force holding the retina in place, thus optimizing the healing benefits of using the proposed system. The magnetic particles can be bio-inert and/or degradable. Other helpful devices are proposed as well to reduce the stresses on the patient's neck muscles in case he/she still needs to hold the head in a certain position for a long period of time.

### CROSS-REFERENCE TO RELATED APPLICATIONS

This present application is based on and is claiming priority and benefits of Provisional Patent Application, Ser. No. 61/601,142, Filed 21 Feb. 2012, Title: “EYES MAGANETICS”, which will be referred to as Ref1, AND this present application claims all the benefits of this Provisional Patent Application No. 61/601,142.

### FEDERALLY SPONSORED RESEARCH OR DEVELOPMENT

Not Applicable.

### SEQUENCE LISTING, TABLE, COMPUTER PROGRAM LISTING, COMPACT DISK APPENDIX

Not Applicable.

### BACKGROUND OF THE INVENTION

### Field of Endeavor

The present inventions relate to the medical field.

They address the issues encountered in the care of the eyes, and specifically in the care of detached retinas.

More particularly, they address certain difficulties encountered when gas bubbles or a silicone oil (GB/SO) are introduced inside the vitreous of the eyeball of the patient and the potential difficulties they could create to the patient, subsequent to the surgery.

Lastly, the present invention describes a number of possible solutions to the above problems.

### Description of Information Known to Inventors

On Jan. 5, 2012, it was determined that my wife, Helma Cherian, had a detached retina in her left eye.

On Jan. 6, 2012, Dr. Mary Hartnett, an ophthalmologist at the John A. Moran Eye Center, in Salt Lake City, operated on Helma, and Dr. Derek Sakata was the anesthesiologist. Dr. Akbar Shakoor assisted Dr. Hartnett during the surgery.

At the end of the surgery, Dr. Mary Hartnett came out of the operating room and talked with me, Gabe Cherian. She told me that the operation went very well. She did not need to use the Belt, which she had mentioned to me before the surgery, that she may need to use, and more importantly, Dr. Hartnett told me that she did not need to take the eye lens out.

Dr. Hartnett also informed me that the tear in the retina was at about 2:00 O'clock.

Finally, Dr. Hartnett told me that she filled the eyeball with Silicone Oil (SI), which will float upwards and will then push the retina against the eyeball wall. This will help in the healing process.

However, in order to let the SI do its job effectively, Helma will now need to hold her head down and to the right, to get the Silicone oil to push against the specific tear location.

Dr. Hartnett wanted us to come back the following day for a follow-up.

To recap:

1. The detached retina had the tear at about 2 O'clock.

2. The surgeons determined that in order to promote the healing, they introduced a certain amount of Silicone Oil (SI) inside the eyeball. The idea is that the SI will float and apply a certain amount of pressure, pushing the retina against the eyeball walls and promoting the healing and the re-attachment of the retina.

3. In addition, the patient was instructed to hold her head in a certain position, to force the SI to be positioned against the proper area of the retina tear.

The effect of introducing the SI inside the eyeball had some positive results, but it has also created a number of undesirable effects.

First, the patient had to maintain her head in a certain position, for several days and possibly for weeks, and possibly in some cases, even up to 3 months, to get the SI to do its job. This meant that during sitting and walking, the patient had to hold her head downwards and tilted to the right. This created a considerable strain on the patient's neck muscles, etc.

In addition, during night sleep, the patient had to sleep in a position, similar to the one maintained during waking hours. She had to maintain that position for a number of days.

All the above creates considerable neck muscles strain and discomfort, during waking hours as well as during sleep.

Second, we understand that the SI has a different Index of Refraction than the normal liquid inside the eyeball. Also, the SI did not fill the entire space inside the eyeball. The effect of all that is that the presence of the SI inside the eyeball has affected the patient's vision's line of sight and the patient's vision was blurry and inconsistently so, as described later under the section on “Problems Review”.

In the meantime, I had a meeting with Dr. Derek Sakata on Jan. 11, 2012. Derek said that he wishes to find an alternative way to help the retina to get attached to the eyeball, instead of using this SILICONE OIL, for possibly up to 3 months, during which the eyesight is very blurry and the patient is under considerable stress and discomfort. So, we started to think and brain storm about something or some solution for this problem.

The standard procedure with this SI is that it should stay in the eye for several weeks at least and possibly up to three months, until the retina looks as if it has re-attached satisfactorily.

However, on Feb. 14, 2012, Dr. Hartnett removed the SI from Helma's eye, by a second surgical intervention. This means that instead of waiting some three months time. Dr. Hartnett felt that it is safe to remove the SI within some five weeks, which made my wife and me happier than if we would have waited longer. At the end of the surgery, Dr. Hartnett came out and told me that the operation worked out OK.

Then I mentioned to Dr. Hartnett that Dr. Sakata and I were talking about any other possibilities to promote the healing of such cases, i.e. like for patients like Helma.

I told Dr. Hartnett about a gadget or device to hold the patient's head in the desired position, to help the patient to hold the head in the desired position, but without straining the muscles. Hartnett agreed that it could be helpful for such patients. It may be a crazy one, but here it is. See FIG. 6. She shook her head, as to say maybe. This FIG. 6 is part of the group of FIGS. 6 through 8. I had showed these to Dr. Sakata in the period between January 8 to 12 or so.

Then I told Dr. Hartnett the following:

How about if we put some magnetic material, e.g. “a MAGNETIC POWDER” inside the eyeball and use a magnet to pull that material against the tear in the retina? Dr. Hartnett said that could be interesting, and it would need some research grant money and then could be tried on animals to prove the concept. She suggested that we could talk more the following day, especially with Dr. Shakoor, who likes to do new things like that. I consented, and we left it at that.

On Feb. 15, 2012, I had dinner with Dr Sakata and I mentioned to him the idea of putting iron powder inside the eye and pull the powder against the retina with magnets placed around the patient's head. He liked the idea and said that we could use Nickel powder, instead of iron powder, because Nickel does not oxidize. Sure, it was a good idea. But Nickel powder can be sharp. I said that we could tumble it, to smoothen out the sharp edges. Yes, he agreed. Then he said that we could experiment with this concept, using the eye of a dead pig. Pigs' anatomy is pretty close to human's anatomy.

We also talked about how to get the magnetic material out of the eyeball, after the treatment is completed. So, we talked about using a needle, with a magnetic tip, which can go in and fish the magnetic material out.

So, again, the magnetic material can be iron powder or nickel powder, and/or any other suitable material that exhibits magnetic properties.

Also the material can be in the nano particle size or whatever size that proves to be suitable. Also, the material can be coated with another biologically inert material. Biologically inert would prevent reactions within the eye such as inflammation.

Later on, Dr. Sakata suggested that we could do something to make the nickel powder “lipophilic”, i.e. more easily attracted to oil, and less apt to stick to the retina or other parts of the eye.

Further later on, the thinking is that the material could be made such it would degrade by itself over time, and would not need to be retracted out by a second surgery.

Further, since the material would be engaged by an externally applied magnetic field, this magnetic field could be intermittently deactivated at times to assess the retinal detachment and reapplied if the detachment required more time with applied force.

On Feb. 20, 2012, Dr Sakata emailed me that he “. . . had mentioned the idea to Dr. Shakoor. Dr. Shakoor stated that the presence of a foreign body with in the eye could be an issue and would have to be removed in the future. In addition, he stated that whatever we chose to use would have to apply equal pressure to a concave surface i.e. the retina and be bio-inert. Bare ferrous material would be an issue. Etc.”.

At that stage, we figured that it is time to document what we have so far. So, this was the reason for the Provisional Patent Application, which was filed on 21 Feb. 2012.

### Prior Art

To our knowledge, there is no prior art teaching about the use of magnetic particles inside the eye for the purpose of promoting the healing of detached retinas.

### REFERENCES

1. Access URL: http://www.researchgate.net/publication/233790258_Biodegradable_nanocomposite_magnetite_stent_for_implant-assisted_magnetic_drug_targeting. Article title: Biodegradable nanocomposite magnetite stent for implant-assisted magnetic drug targeting; By Jan O Mangual, Shigeng Li, Harry J Ploehn, Armin D Ebner, James A Ritter, Journal of Magnetism and Magnetic Materials (impact factor: 1.78). 10/2010; 322(20):3094-3100. DOI:10.1016/j.jmmm.2010.05.036.

2. Access URL: http://www.cnsi.ucsb.edu/events/2011/sailor/. Seminar title: Biodegradable Nanoparticles Based on Silicon and Iron Oxide for In-vitro and In-vivo Applications; Speaker: Michael J. Sailor, Department of Chemistry & Biochemistry, Department of Bioengineering, Department of Nanoengineering, University of California, San Diego; Date & Time: Jan. 21, 2011, Noon-1:00 pm, 1605 Elings Hall.

3. Access URL: http://pubs.acs.org/doi/abs/10.1021/nn103213y. Article title: Mesoporous Biocompatible and Acid-Degradable Magnetic Colloidal Nanocrystal Clusters with Sustainable Stability and High Hydrophobic Drug Loading Capacity; by Bin Luo†, Shuai Xu†, An Luo‡, Wen-Rui Wang §, Shi-Long Wang §, Jia Guo†, Yao Lin⊥, Dong-Yuan Zhao∥, and Chang-Chun Wang†*

4. Access URL: http://en.wikipedia.org/wiki/Iron_oxide_nanoparticles. Article title: Iron oxide nanoparticles; From Wikipedia, the free encyclopedia. Under “Biomedical Applications”, the article states: “Magnetite and maghemite are preferred in biomedicine because they are biocompatible and potentially non-toxic to humans^{[citation needed]}. Iron oxide is easily degradable and therefore useful for in vivo applications^{[citation needed]}.”

5. Access URL: http://www.ncbi.nlm.nih.gov/pubmed/20820094. Article title: “Degradability of superparamagnetic nanoparticles in a model of intracellular environment: follow-up of magnetic, structural and chemical properties.” Lévy M, Lagarde F, Maraloiu V A, Blanchin M G, Gendron F, Wilhelm C, Gazeau F. Source: Laboratoire Matière et Systèmes Complexes, UMR 7057, CNRS and Université Paris Diderot, Paris, France. Nanotechnology. 2010 Oct. 1;21(39):395103. doi: 10.1088/0957-4484/21/39/395103. Epub 2010 Sep. 6.

6. Access URL: http://www.nano-reviews.net/index.php/nano/article/view/5358/6032\NANO REVIEWS. Article title: Review Article—“Potential toxicity of superparamagnetic iron oxide nanoparticles (SPION)”. Neenu Singha, Gareth J. S. Jenkinsa, Romisa Asadib and Shareen H. Doak; Institute of Life Science, School of Medicine, Swansea University, Singleton Park, Swansea, Wales, UK; Cardiff School of Biosciences, Biomedical Sciences Building, Museum Avenue, Cardiff, Wales, UK. Received: 6 Jun. 2010; Revised: 2 Jul. 2010; Accepted: 9 Jul. 2010; Published: 21 Sep. 2010.

### BRIEF SUMMARY OF THE INVENTION

### Problems that Seem to Need Attention and Solutions

Mainly, they are problems related and/or caused by the introduction of Gas Bubbles and/or Silicone Oil (GB/SO) inside the eye. This creates:

a) the need to position the eye and the head in certain positions, for extended length of time, which results in muscle tension and stress,

b) possible blurriness of vision, and lastly

c) whether the use of the GB/SO addresses all cases of retina detachments, including the case where the retina detachment happens to be in the lower part of the eyeball.

### OBJECT OF THE INVENTION

a. The general objects of the invention are to provide alternative solutions, which can promote the healing of torn retinas, after the completion of the conventional retina re-attachment procedures.
b. One specific object is to use magnetic materials, including degradable magnetic materials, together with magnetic fields, to provide a more controllable pressure on the retina, for the same purpose of promoting the healing process, even if the patient does not comply with the request of maintaining his/her head in the recommended position(s) and actually does move his/her head; and also in the case of when the tear of the retina occurs in the lower part of the eye,
c. Another object is to provide solutions for the case, where the patient does actually move his/her eye only, even though he/she does not move his/her head.
d. In other words, the object is to provide solutions to maintain the proper pressure on the retina, i.e. the pressure that is desirable to exist between the retina and the eyeball walls, to promote the healing and the re-attachment of the retina, even if the patient does move his/her head and/or does not maintain his/her head in the recommended position. In other words, our proposed solutions would not depend on the force due to the difference in the specific density of the SI and the liquid in the eyeball, but it would depend on the force resulting from the use of the more controllable effect of the interaction between the magnetic field and the magnetic particles, regardless of the position of the patient's head or the absolute pressure inside the patient's eye, or on the altitude of where the patient lives, etc.
e. Another objective is to provide a dynamic system for applying pressure to the healing retina such that the force can be changed and/or discontinued, to check the healing, and/or to be reapplied if pressure needs to continue.
f. Another object is to reduce the muscle pain and stress, which result from the need to maintain the patient's eyes and/or head in certain positions, for extended length of time, by providing certain devices to help the patients in positioning their head in any certain desired position, if this is still necessary.
g. So again, the general objectives of all this is to provide methods and means to help in promoting the healing of detached retinas, as well as to reduce the resulting stress, pain and suffering of the patients, again after the initial surgical intervention.
h. The possible additional consequences of all the above is to reduce the time and financial burden on the patients and on the professional medical staff caring for these patients.

It is also possible that the inventions covered by this application may also be applicable to other similar situations, wherever applicable.

We can think about back surgeries, abdominal surgeries, mouth or teeth surgeries and the like. The idea is to utilize external energies, with or without additional invasive means or procedures, to promote the healing after the initial surgeries have been done, or perhaps even before invasive surgeries, thus reducing some of the possible complications that may result from such surgeries.

These solutions as well as their benefits and advantages will become clearer when we describe the drawings in detail, further down below

### Brief Review of the Problem and the Proposed Solutions

Silicone Oil and Gas Bubble Problems (GB/SO); Blurry Vision:

One of the problems that could occur due to the presence of the Silicone Oil inside the eyeball, or even Air Bubbles, which is another alternative to using Silicone Oil, is blurriness of vision.

We can easily visualize the potential effect of such foreign materials inside the eye, which can happen especially if and when such materials move and position themselves across the light path between the eye lens and the retina, especially the focal area of the retina. See FIG. 1, which shows the path of lines of vision as (131). These foreign materials have refraction or diffraction indices that most probably are different than those of the natural liquids inside the eyeball. If such materials get in the path of the light (131) between the eye lens and the visual focal area of the eye, then the light will be diverted and the image on the retina will be distorted. It can even be possible that these foreign materials could totally block the light rays from reaching their destination.

We can easily visualize that the minimum effect for such intrusion on the light path would be a blurry vision, in a way similar to the case when a person is swimming under water and open his eyes in the water, without proper eye masks or goggles. The water would create a blurry vision of some sort, with possible unsteady or wavy view, due to the ripples of the water, even if the water is crystal clear without any particles floating in the water. We can also visualize that if the silicone oil or the air bubbles happen to be in the line of sight (131) inside the eyeball, they could have at least an effect similar to the one just described here

Worst than that, is that this blurriness can be inconsistent or not repeatable. It is possible that the blurriness can change, depending at least on the position and movement of the eye. If the eye is positioned just right, then the SI will float away from the direct line of sight, i.e. from the eye lens to the focal area. In this case, the patient will not perceive any ill effects. But if the position of the head or the eye changes and is such that it allows or forces the SI to move and to come between the eye lens and the eye sight focal point, then the SI will create a condition as described above and the patient's vision will become blurry. So, this inconsistency may be even more disturbing than otherwise. The patient will not have any confidence as to the reliability and repeatability of his/her eye sight and that can be as bad and as disturbing, and even worse than having blurriness all the time.

Silicone Oil and Gas Bubble Problems; Position of the Eye and of the Head:

In order to maintain the effect of the SI and/or gas bubble and to maintain the pressure of the SI on the retina and to promote the healing, the patient is advised to maintain the eye and the head in a certain position. The desired position could require that the patient tilts his/her head into an awkward unnatural position or angle, which in turn can generate a lot of muscle tension and stress on the neck, shoulders, etc. This can be especially annoying and distressing if this desired position needs to be kept for extended length of times.

Silicone Oil Potential Problems: Silicone Oil and Radiation. If a person, with such silicone oil introduced in his/her eye, gets exposed to radiation of one sort or another, could the eyeball explode? Any previous experience with such situations? Have there been any tests done regarding this eventuality?

### BRIEF SUMMARY OF THE INVENTION

We propose the following solutions:

1. The use of bio-inert, magnetic particles or materials and ancillaries. See FIGS. 1 through 5.

2. The use of electro magnets, together with eye monitors, to accommodate patients' eye movements. See FIGS. 1 and 5.

3. The use of “DEGRADABLE MAGNETIC” materials, to avoid a second invasive procedure.

4. The use of a certain positioning caddy, to support and restrain the head movement. See FIG. 6.

5. The use of certain support pillows and cushions, with controllable pressure distribution. See FIGS. 7 and 8.

### Advantages of the Proposed Solutions

1. The bio-inert, magnetic particles or materials can be controlled and positioned in a way, so that, under the influence of the applied magnetic field, the magnetic particles will stay in the proper place and location, against the repaired area of the retina, and will apply the desired proper pressure on that area, so as to promote the healing process. This will happen, regardless of whether the patient will move his/her head in different directions, and regardless of the effect of the natural gravitational forces. Also, the same will happen regardless of the location of the tear in the retina, i.e. whether the tear is in the upper part of the eyeball or in the lower part. Also the same advantage will be obtained regardless of the intraocular pressure (IOP) inside the patient's eye.

The present conventional approach to treat such retina tears is obviously to first repair the tear, and then to inject either Gas Bubbles or Silicone Oil (GB/SO) inside the eyeball.

I am not an ophthalmology doctor, not even a medical doctor. I am, however, a mechanical engineer. I rely to a large extent on information I collected during my contacts and interaction with the expert doctors, while trying not to be too intrusive and bothersome. I augment my knowledge by scanning and reading some of the available literature. So I am basing most of my thinking on that and on the advice of Dr. Sakata. But I am sure that I do not understand all that there is to it, and I have certain questions.

The conventional theory is that the GB/SI are lighter than the liquid inside the eyeball and will float on top of the eyeball liquid and thus will apply a certain amount of pressure on the retina, at the location of the repaired tear, and to push the repaired portion of the retina against the wall of the eyeball and thus would promote the re-attachment of the retina to the wall and promote the healing process.

This conventional theory may work very well in certain cases, but in other cases, it may not work as well. The reason is the following.

The theory assumes that the tear in the retina happened at the upper part of the eyeball, so that the lighter GB/SI will float upwards and would reach the area of the tear and would apply the desired pressure, etc. However, there are the following cases, where this theory may not apply or may not work as expected and as hoped.
a. I do not really understand why we need to inject any additional material inside the eyeball, such as the conventional GB/SO. Is it perhaps because the liquids inside the eyeball do not totally fill the eyeball space and that there is an empty space of some sort inside the eyeball that may leave the repaired portion of the retina hanging down, unsupported? If this is not the case, then why can't we simply rely on the existing eyeball liquid and the existing Intra-Ocular Pressure (IOP) to do the same desired job, as the one expected from the injected GB/SO?
b. If the theory is to simply increase the total pressure inside the eyeball, then in this case, we may get the benefit of the additional pressure to push the retina back in place. However, there is another side to this coin. If we increase the total pressure too much, this may be conducive to getting glaucoma. If this is true, then what?
c. The other possible scenario is if the IOP gets too low, for one reason or another. Then, the pressure applied on the repaired retina may not be sufficient to incur the desired healing effect.
d. The patient is advised to hold his/her head in a certain position, so that the natural force of gravity would move the GB/SO against the area of the repaired retina and would apply the desired push on the retina at that desired location. Requesting that the patient maintain this position is a problem. If the patient complies, then he/she may get a stiff neck and/or other troubles. If the patient does not comply, then we loose the benefits of the injected GB/SO.
e. How about if the patient lives in a place at a high altitude. Would the lower force of gravity extend the duration of the healing process? Or even totally negate the whole purpose of the effort?
f. What if the tear happens to be in the lower part of the eyeball? We are not sure how frequently this condition does happen, but if it does, then this conventional approach may not be as effective.
g. It seems that the theory of injecting the GB/SO relies on the difference between the specific densities of these GB/SO and of the eyeball liquids, and that the hope is to place these GB/SO against the torn retina. So, if the tear of the retina is at the lower part of the eyeball, these GB/SO would not reach the torn retina area. Would this reduce the effectiveness of the conventional approach?

So based on the above, here is what we think are the advantages of our proposed approach.

If the tear in the retina is in the upper half of the eyeball, then the magnetic field acting from the top of the head, by the magnets (123), (223) and (333) in FIGS. 1 through 3, or by the magnets M1, M2, . . . through M5 in FIG. 4 or by the magnets N1, N2, . . . through N7, in FIG. 5, will pull the magnetic material upwards, counteracting the effect of the natural force of gravity and will push the magnetic material against the retina and consequently and in turn, it will push the retina against the eyeball wall, with the hope that this would promote the re-attachment of the retina to the eyeball and would promote the healing process.

If the retina tear happens to be in the lower part of the eyeball, then the magnets would be placed below the eyeball, as in FIG. 5, by magnets S1, S2, . . . through S7, which could be placed say around the neck, supported by collar carrier of some sort and we would get the same end effect, i.e. we would still get the same healing process improvement.

In both cases, the pressure inside the eyeball does not need to be elevated or changed for any related reasons.

So, we will not rely anymore on the force due to the difference in density of the GB or the SI and the liquid in the eyeball, but we will have a controllable force, which is created by the strength and direction of the magnetic field and its effect on the magnetic particles inside the eye.

The magnetic particles and the magnetic field will fight and counteract the natural force of gravity, and the magnitude and direction of the created applied force of the magnetic particles will be optimized and directed to be in the most desirable location and direction, and the particles will stay and remain where we want then and need them to be.

2. The monitoring of the eye movement by the eye sensor (251) in FIG. 2 and (551) in FIG. 5 is an additional feature to make sure that the magnetic approach is in full effect even if the patient moves his/her eyes, even when his/her head did not move at all.

3. Most of the time, with the present conventional methods of using Gas Bubbles or Silicone Oil, there is a need for a second surgical intervention to remove the remaining gas bubbles or the silicone oil out from the patient's eye.

In contrast, we propose to use “DEGRADABLE MAGNETIC MATERIALS” for our “magnetic” approach. We hope that by using such degradable materials, we would most probably be able to eliminate the need for a second surgical intervention. This would minimize the pain and suffering of the patient, and would free up some time to the medical staff to address the needs of other patients and would reduce the total cost of medical care in general.

4. The head restraint and support in FIG. 6 can be used if and when the proposed magnetic approach is not use and adopted.

5. The support pillow and cushions in FIGS. 7 and 8 can be used in either case, i.e. with or without the magnetic particles approach. It will give more comfort to the patient.

### Definitions and Synonymns

AC=Alternative Current

DC=Direct Current

GB/SO=SO/GB=Gas Bubbles or Silicone Oil

IOP=intraocular pressure

SI=Silicone Oil

wrt=with respect to

### BRIEF DESCRIPTION OF THE SEVERAL VIEWS OF THE DRAWINGS

FIG. 1 shows the general scheme of the invention, showing a simplified cross-sectional view of a patient's eyeball, highlighting the detachment area (111) of the retina, and the proposed magnet (123) placed adjacent to the head and the eye, and the proposed magnetic material (121) or (127) that would be inserted into the eyeball. The magnetic material is shown in two possible positions. First, (121) at the bottom of the eyeball, when the magnetic field is not activated. Second, (127) near the top of the eyeball, near the area where the retina was torn and was repaired, and when the magnetic field (125) is on and has pulled the magnetic material against the retina, pushing the retina against the walls of the eyeball, for the purpose of promoting the re-attachment of the retina and the healing process.

The figure also highlights the fact that in either of these two positions of the magnetic material, the magnetic material will stay outside the line of sight (131) of the patient, so the patient will not experience any vision blurriness, in either situation.

FIG. 2 shows a simplified side view of a patient's head, while wearing a carrying device, say a head gear (241, 243), which in turn carries the activating magnet(s) (223). When activated, the magnet will induce a magnetic field (225), which will pull the magnetic material towards the magnet as shown in FIG. 2 and in so doing, the magnetic material will be pushed against the retina, at the spot that was previously surgically repaired and by doing so, the retina will be pushed closer against the walls of the eyeball, thus promoting the re-attachment of the retina and the healing process.

FIG. 2 shows also an eye movement sensor (251), which can be placed in a way so as to monitor the patient's eye movement. If any movement is detected, even if the patient's head has not moved at all, then the sensor would send a signal so that the magnetic field can then be adjusted to ensure that the magnetic material will still provide the optimum desired pressure on the retina as planned. See also FIGS. 4 and 5.

FIG. 3 shows a front view of the setup in FIG. 2.

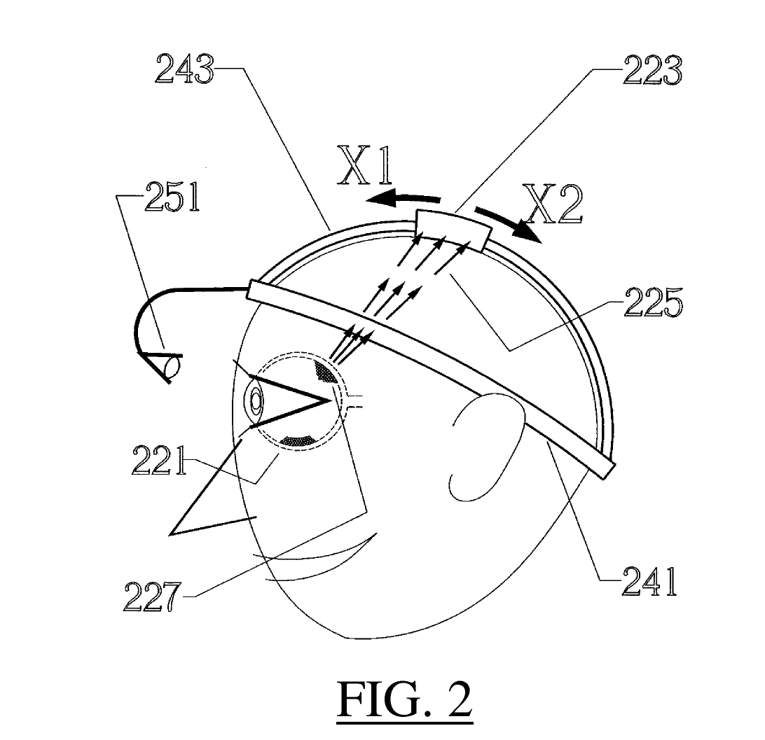

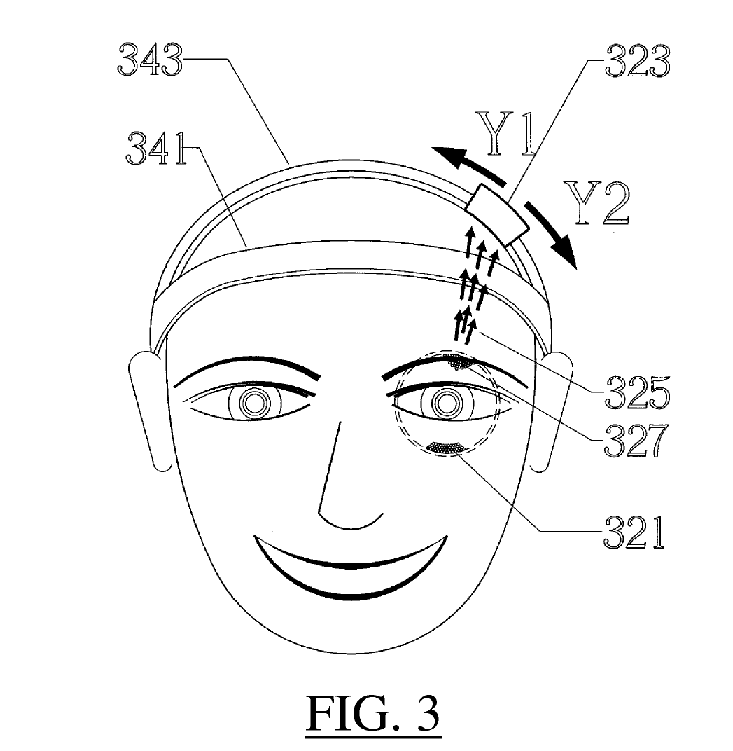

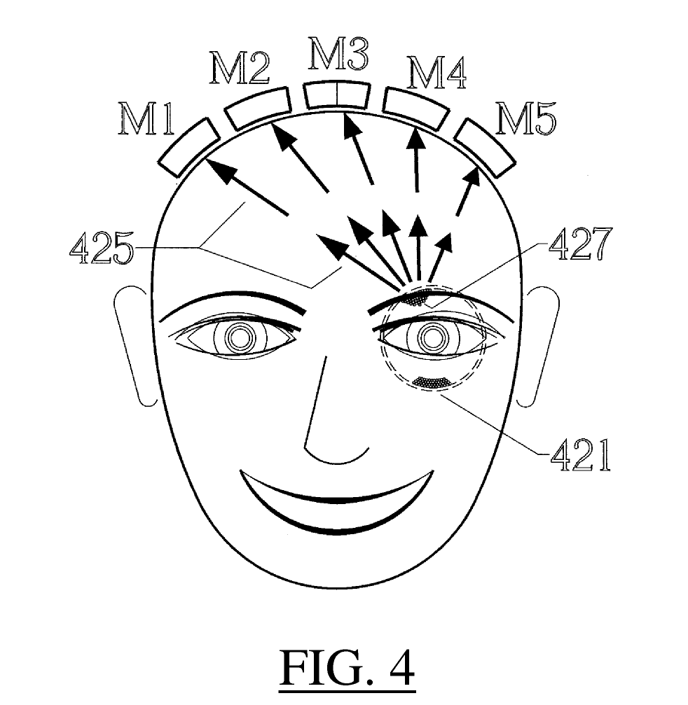

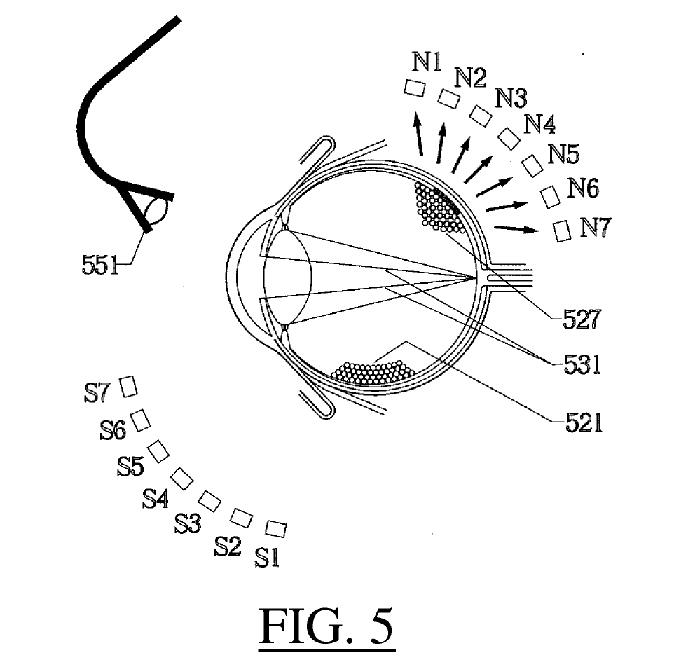

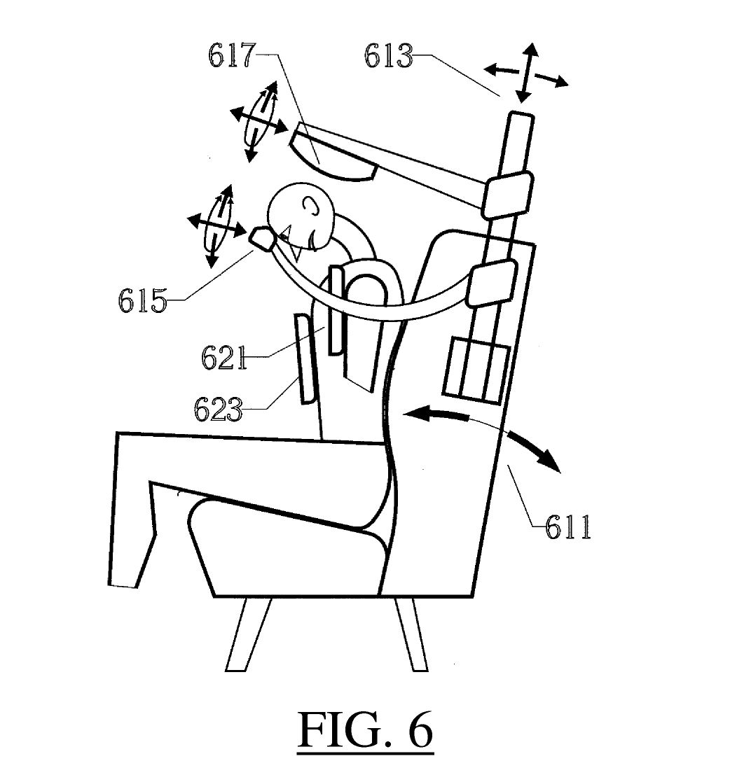

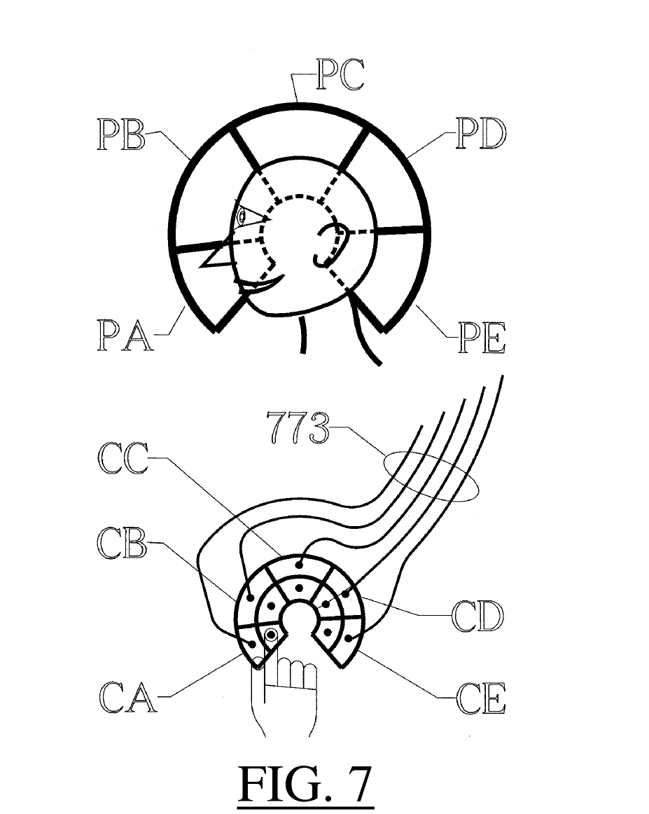

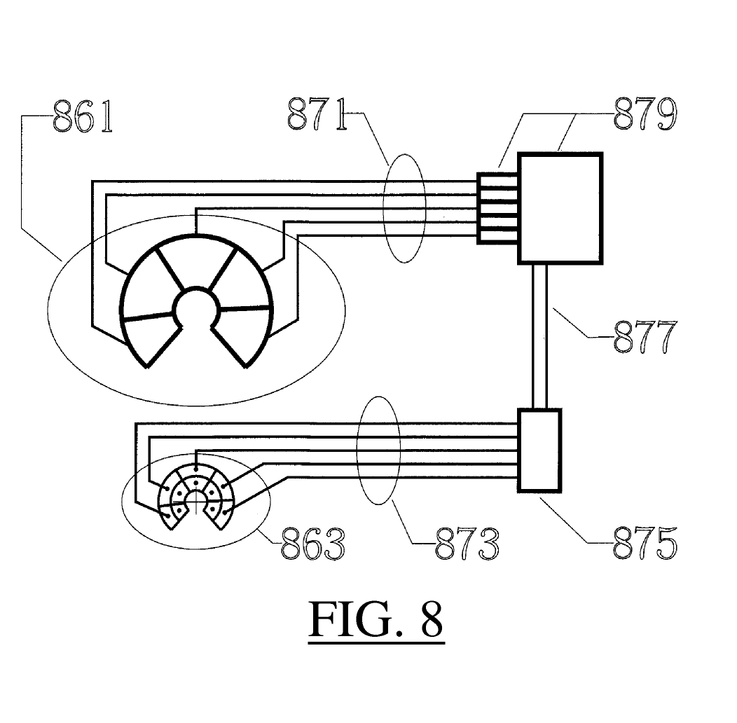

FIGS. 2 and 3 further show that the magnet (223) or (323) can be relocated and moved in an X1, X2 direction as in FIG. 2 and/or in a Y1, Y2 direction as in FIG. 3, along the head gear (241), (243), or (341), (343) or along one of its straps, to optimize the direction of the magnetic field (225), (325), depending on the location of the retinal tear.

FIG. 4 shows that we can have more than one magnet, as illustrated by M1, M2, . . . through M5, and that we can place them such as to optimize the magnetic field 425, to optimize the pull force and its direction.

FIG. 5 shows that we can have more than one set of magnets, Set 1 comprising N1, N2, . . . through N7, and Set 2 comprising S1, S2, . . . through S7, and that we can arrange them in different ways, again to optimize their effect.

FIG. 6 shows a support and restraint arrangement, to reduce the pain and stress on the patient if and when the patient is required to keep his/her head in a specific position. The support (615) can be made to have some foldable sections or edges to create a sort of a cradle and can also be rotated axially to optimize the way it keeps the patient's head in the rest position.

They can be adjusted in their height, in the distance between them, and they can be rotated axially as well, in order to attain the most comfortable position for the patient. They can be mounted on a stand-alone stand (613), or on a wheel base, etc. they can be constructed so that they can be attached to a bed, to an arm chair (611), or to a wheel chair, etc.

FIGS. 7 and 8 show a multi-compartment, inflatable cushion, which can comprise several compartments, such as PA, PB, . . . through PE, which can be used by a patient, who is required to keep his/her head in a certain position. Actually it can be used by any person, simply to have a more comfortable sleep or rest.

FIG. 7 shows the cushion itself, with its compartments PA, PB, . . . through PE, and its control device, with its control elements CA, CB, . . . through CE, and FIG. 8 shows some details of the connection between the cushion and the control.

This embodiment can also be incorporated in the embodiment shown in FIG. 6 as well.

### DETAILED DESCRIPTION OF THE INVENTION

While the invention is susceptible to various modifications and alternative constructions, certain illustrated embodiments thereof have been shown in the drawings and will be described below in detail. It should be understood, however, that there is no intention to limit the invention to the specific form(s) disclosed, but, on the contrary, the invention is to cover all modifications, alternative constructions and/or methods, and equivalents falling within the spirit and scope of the invention as defined in the claims.

While we are describing the drawing in more detail, we will at the same time explain the technological basis of the invention. I will also include a number of examples in this section, which should be considered as part of the embodiments for the purpose of this application as well.

This description covers more than one invention. The inventions are based partly on the same technological platform, but then each of the inventions has some additional features of its own. We would like to leave it to the patent examiner to decide on the number of the inventions contained and how to split one invention from the other.

Finally please note that the dimensions, especially the size of the particles and of the magnets and other components and elements, shown in the various figures, are arbitrary and not to any special scale. They just illustrate the design concepts. The actual dimensions of any of the devices, according to the shown embodiments, should be chosen to suit the specific respective application case.

### First Preferred Group of Embodiments

1. The Use of Magnetic Particles or Materials and Ancillaries.

1. Magnetic Material Inside the Eye and Controlling Magnets Outside:

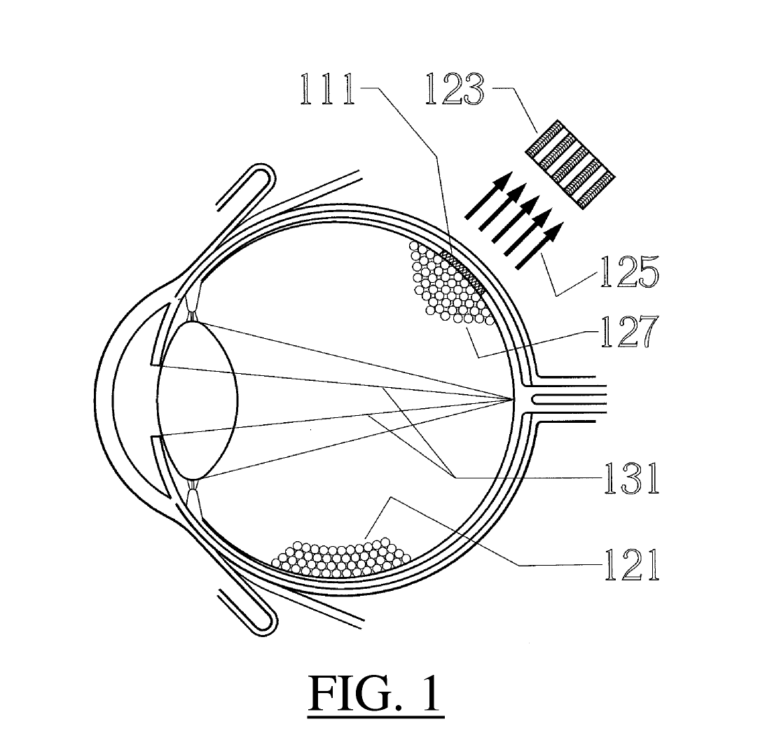

FIGS. 1 through 5 cover the concept of the magnetic material inside the eye.

FIG. 1 illustrated the concept of using an external magnet (123) and some magnetic material (121) or (127) that could be inserted or injected inside the eyeball. The magnet will attract the material towards it, as in position (127), thus forcing and/or pulling the magnetic material in its direction. Depending on the position of the magnet with respect to the eyeball and wrt to the tear (111) in the retina, the magnetic material would apply a certain amount of force against the retina, encouraging the retina to adhere more closely to the eyeball walls, in that area and in that direction. By properly positioning the external magnet around the patient's head, we can control the direction of the magnetic material inside the eyeball, to accomplish our goal.

The strength of the magnetic field (125) and its direction will affect the outcome. FIGS. 2 and 3 show the external magnet (223) and (323), and how it can be strapped around the patient's head to accomplish our desired and stated goals. We can use a head fixture or head gear, (221) and (243), or (341) and (343), similar to many of the various head gears used in sports, to protect athletes or bikers for example, or similar to the head straps used to attach a CPAP face mask to a patient's face.

FIG. 4 shows that we can use a number of magnets, e.g. (M1), (M2), . . . through (M5), in our effort to control more precisely the location of the material (427), inside the eyeball and to control its distribution and to promote its adherence to the “curvature” of the eyeball. So, for example, Magnet M1 would have its flux pulling towards the left of the figure, in the direction shown in FIG. 4, and Magnet M4, will have its flux in the direction shown, pulling towards the top or slightly towards the left of the figure, etc. We can control the number of magnets, as well as their magnetic strength etc to create a 3-D pattern of the magnetic material inside the eyeball and to encourage it to follow the internal topography of the eyeball.

### Electro Magnets

The above magnets can be as “permanent” magnets. On the other hand, they can even be electromagnets, with coils, etc, so that we can even more precisely control the strength of the magnetic flux and its direction and how it would encourage the magnetic material inside the eyeball to do its intended job. They can be small in size, light weight, adjustable within the head gear, in X or Y directions or in polar coordinates directions.

FIG. 5 shows yet another embodiment of the idea/concept. We can have the magnets split between two poles, as N1 and S1, i.e. the North pole, N1, of the first magnet, work together with its South Pole, S1, and then N2 working with S2, and so on. We can thus better control the power and the power distribution and the direction of the flux lines, to achieve the optimum position and spread of the magnetic material inside the eye, to obtain the best end results.

The magnets shown in FIG. 5 can be at the shown locations either permanently, or they can be there only during the initial set up and then they can be replaced by magnets as in FIG. 4.

Also all these magnets, can be one single magnet or multiple magnets. And they can be “permanent” magnets and/or “electro-magnets”, i.e. magnets that are operated by an external electric power. And they can be operated by batteries or any other kind of electric power.

Electro magnets can be powered by an electric power source, which could provide either a DC or an AC voltage and current. They can be turned on and off purposely and/or periodically, at will, for a number of reasons.

1) To make sure that the magnetic particles do not stay in the same position for an excessive length of time, otherwise they may “stick or lump” together and/or may “permanently” stick to one position near the retina in one position for a long period of time, and may become too difficult to entangle and dislodge when we do want to move them e.g. when the time comes to remove the particles from the eyeball.

2) To allow the particles to be relocated from one particular area or spot along the retina to another spot, and/or if we want them to accommodate the movement of the eyeball, when the patient does move the eye out of its normal rest position. So, we could turn the power off so that the magnets will release their hold on the magnetic particles and let the powder settle down or at least becomes fluffy, if you will, and then after a certain length of time, which will be determined by proper experimenting, we would turn the power back on to re-energize the electro-magnets to get the magnetic particles to move back against the retina and for them to resume their effort to enhance the re-attachment of the retina to the eyeball. This can be done purposely, as will be discussed further in the next section down below, under “accommodating for eye movement”.

3) To allow intermittent release of pressure to check if the retina requires continued pressure to heal.

Improved Effect of Magnetic Particles in Reducing or Eliminating Vision Blurriness:

If we use the proposed magnetic particles, instead of the conventional silicone oils or air bubbles, then we would have less chance of getting the particles in the path of light between the eye lens and the focal area of the retina. This path of light is exemplified by the lines (131) in FIG. 1 and lines (531) in FIG. 5.

We would expect to have either one of the following two conditions. One condition is when the magnets are operational, and the second is when the magnets are idle or removed.

Please refer to FIGS. 1 through 5.

In the first case, when the magnets are operational, the magnets would be pulling the particles elements (127, 227, 327, 427 and 527) against the area(s) of the retina (111) in FIG. 1, which were torn and have been repaired and which are, hopefully, far enough from the focal area of the retina. This will ensure that the particles would not be within the line of sight (131 and 531) in FIGS. 1 and 5. This is illustrated in FIG. 1, in the upper right corner of the eye, where the magnetic particles (127) have been gathered fairly close to the repaired retina (111), by the magnets (123).

In the second case, when the magnets are not operational, the magnetic particles, which are expected to be have a higher density than that of the eyeball liquids, will settle down near the bottom of the eyeball, as shown by (121, 221, 321, 421 and 521) and thus will again be away, out of the way from the line of sight. This is illustrated again in FIGS. 1 and 5 by the lines (131 and 531), but it is shown in the lower part of the eye, where the magnetic particles have fallen, under the force of gravity, to the bottom of the eyeball.

Thus, in either case, the magnetic particles would be out of the way from the line of sight of the patient and we would expect that they will not create any blurriness or undesirable effect to the patient's vision.

This turning on and off of the electro magnets should ideally be done under an expert eye doctor, and while the doctor is looking at the inside of the eye with his regular proper instrumentation, which are used to examine the insides of patients eyes.

### Second Preferred Group of Embodiments

2. Degradable Magnetic Materials

As mentioned above, under the section on “ADVANTAGES OF THE PROPOSED SOLUTIONS”, one of our hopes and goals is to find a way that would avoid or eliminate the need for a second surgical intervention, which would most probably be required, to remove the magnetic material, which was injected into the patient's eye during the first surgery, if we had used regular magnetic material during that first surgery.

We feel that if we use a magnetic material that is degradable over time, then we would accomplish this goal and we would not need any second surgeries.

So we feel that the next phase should be to try to find a DEGRADABLE MAGNETIC material, which can do a similar function as the above magnetic particles, but which would degrade and disappear as time goes by, which would mean that we, most probably, would not need to go back into the eye and try to remove the material, using a second surgical intervention.

But instead of re-inventing the wheel, we did a literature search. It revealed that a number of articles, covering the subject of such degradable magnetic material, have been published already. We have listed six of those articles under the section on “REFERENCES” above, just as examples of such articles.

Some of the published articles are very encouraging, while a few articles warned about some negative aspects and after-effects, such as toxicity.

For this reason, we feel that we need to be extra careful in choosing the best possible material for our proposed approach.

So, we will proceed with this phase of our proposed approach, taking all the precautions necessary to ensure that we end up with a safe and effective and acceptable solution.

We suspect that in order to find such an acceptable degradable magnetic material and to prove its efficacy, it would require a considerably longer time to find a good acceptable material or materials. So, in the meantime, we would proceed with materials that are already known presently, but we should expend certain effort towards finding the desirable degradable replacement material as well.

### Third Preferred Group of Embodiments

3. Electro Magnets and Accommodating Eye Movements

Please look again at FIGS. 2 and 3, and especially at FIGS. 4 and 5.

The electro magnets can also be controlled by a special program, e.g. computer programs and the like.

We can use this fact in adjusting the magnetic field to accommodate for eye movements:

The patient naturally can move his/her eye in several directions, even without moving his/her head. This may not be too bad and may not have any drastic effect on the pressure that the magnetic particles are exerting on the retina. However, depending on proper research, it may become desirable to adjust for such eye movements, to maximize the effect of the magnetic particles.

A way to do that is the following.

We can use a sensor such as (251, 551) in FIGS. 2 and 5, to detect the eye movement with respect to the head position. Such a sensor can be similar to those offered to be used, for example, to detect whether a truck driver is getting drowsy or sleepy, or to monitor the eyes of airplane pilots.

The sensor can be mounted on a temporary or rather a detachable connection, which can be part of the head gear, which carries and controls the electro magnets. This is illustrated in FIG. 2. The sensor(s) could also be mounted on eyeglasses, which are already being used by the patient, or on temporary eyeglasses frames, used just for this purpose. When the patient goes to sleep or does not want the sensor for a certain length of time, then the sensor can be unplugged and detached from its temporary support and put aside.

But when the sensor is in place and is active, then the sensor will monitor the eye movement and when the movement of the eye, with respect to the head/skull becomes larger than say a certain predetermined limit, then the sensor will send a proper signal to a control system, not shown, which can be part of the head gear, for example, wherein the control system will turn some electro magnets off, and would turn some other ones on, or simply change the electric inputs to the magnets, so as to alter the magnetic field and the attraction force, which affect the position of the magnetic particles, so as to optimize the location and direction of the pull force to the more appropriate conditions, so that the effect of the magnetic particles stay at an optimum level.

Here is another way to explain the purpose of this proposed embodiment. Say the magnets and the magnetic field have been set-up initially, with the position of the eye and of the tear in the retina at a certain initial position with respect to, or relative to, the patient's head/skull.

Then, suppose that the patient moves his/her eyes to a new position, with respect to his/her head/skull, even though he/she did not move his/her head at all. In this case, the eye sensor monitoring the eye, would detect this relative eye movement with respect to the head/skull and its original setting. If and when the new position is considerably different than the initial one, and based on certain predetermined criteria, then the eye sensor and its respective control unit will trigger the magnets control system to implement the necessary corrections of the magnetic field pattern, so that the new magnetic field configuration would re-establish and maintain the desired pull on the magnetic particles in the desired position and direction, against the repaired retina area.

All this can be controlled by appropriate algorithms that can be developed specifically for such a device and such a method, with the goal of optimizing the effect of the system. We are sure that anyone skilled in the art can compile such a control program.

### Fourth Preferred Group of Embodiments

### 4. Positioning Caddy

FIGS. 6 through 8 cover the concept of a device to hold and support the patient's head in a certain position, while awake and while sleeping.

FIG. 6 shows an external device to help in positioning the patient's head in the most desirable position, to promote the healing of the eye, after the surgery, yet at the same time, it help in reducing the pain and stress on the patient's neck and shoulders muscles.

This is especially important if the magnetic particles approach is not used.

FIG. 6 shows a support member (615) and a restraining member (617), which we will call PILLOWS.

One of the pillows (615) can be placed below the patient's head, referred to as the “REST or SUPPORT”. The other pillow can be placed above the head, with some free space clearance as shown. This second pillow (617) can be called the “Position Control” pillow or the “RESTRAINING” pillow. We should leave enough clearance between the restraining pillow (617) and the patient's head to allow the patient to breath etc. Especially, to avoid the feeling of claustrophobia.

We may also place these pillows on an angle, as needed, to achieve the best position of the head and eye, which best promote the healing process.

This Positioning Caddy can be as a stand-alone device, or can be on an independent support or stand, that can be moved along and/or can be attached to a chair, arm chair, wheel chair, bed or the like. Similar to the supports or stands that are used with patients beds in hospitals for example, and which carry the intravenous feeders of medicines infusion or nutrients liquids, as part of the patient care. When the patient needs to get out of the bed or off the chair, e.g. to go to the bathroom or the like, then this support can be attached to a wheel chair for example. Also when the patient is allowed to get out of bed and walk around for a while, the same device can be carried by a stand, that is mounted on a wheel base, again like those units frequently used in hospitals, so that the patient can hold on to it and pulls it along, while walking. In both cases, the device becomes an “ambulatory” device, at least for a while.

The cushions in this embodiment can also be inflatable and controllable, similar to the ones in the fourth preferred group of embodiments, as described down below.

Additional supports or cushions can be provided for the patient's comfort. For example, a support (621) for the arms/shoulders, or another support (623) for the chest, can be provided and adjusted as necessary.

### Fifth Preferred Group of Embodiments

### 5. Support Pillows and Cushions

FIGS. 7 and 8 show another embodiment of an external device to help in positioning the patient's head in the most desirable position. Again, this device helps in reducing the pain and stress on the patient's neck and shoulders muscles. It is a pillow, with various partitions or compartments, PA, PB, . . . through PE, in FIG. 7 and with controllable pressure inside the individual compartments. The idea is to use such a pillow to better position the patient's head, while laying down or sleeping.

This can be used in either case, whether the magnetic particles approach is used or whether the traditional air bubbles or silicone oil approaches are used.

FIG. 7 shows the concept of the inflatable pillow, which could be shaped like a horse-shoe like tube, where the circumference of the pillow is divided into a number of segments or compartments, PA, PB, . . . through PE. Each one of the compartments is connected to an air pressure supply, see (879) in FIG. 8. The lower parts of the figures, FIGS. 7 and 8, illustrate the control device, item 863 in FIG. 8, which can comprise its respective control buttons or control elements, CA, CB, . . . through CE, which would allow the user to control the air pressure and volume in each one of these various pillow's compartments. Thus we can control the position and the comfort level of the patient.

The cushion or pillow can comprise a number of individual compartments, e.g. PA, PB, . . . through PE, where each compartment can be inflated to a desirable pressure and desired shape. The pressure can be regulated by a control device, which would comprise means to control and adjust the pressure in each compartment, say by pushing on a certain button, e.g. CA, CB . . . through CE, each button relating to one respective compartment of the pillow.

A cable, (773) in FIG. 7, or the elements (873), (875), (877) and (871) in FIG. 8, connect the control unit to the pillow, and to a pump and related valves (879) shown in FIG. 8.

FIG. 8 tries to explain how each compartment of the pillow shown in FIG. 7 can be wired and can work.

The pump and related valves (879) provide the compressed air to the cushion and to its individual compartments, and the connector (875) facilitates attaching the control unit to the pump and cushion.

A similar approach of using controllable pressure distribution as described for this embodiment can be used for the embodiment shown in FIG. 6 as well.

### Special Notes and Considerations

The above proposed procedures, i.e. the use of the magnetic particles and magnets are serious matters, and should be given serious safety considerations. We would like to point out at least the following important points.

A lot of preparatory research and experimental work need to be done and repeated and verified etc before any of the proposed concepts are applied to, and used on, humans. We are sure that the medical professionals who will be interested in pursuing these proposed concepts are conscientious enough to do the right things.

Some of the issues that are of concerns are discussed below, but we are sure that the list does not cover every conceivable possibility. Here are a few that come to mind at present.

The tools which can be used to inject/introduce the magnetic particles inside the eye and then to extract these particles, once they have accomplished their job, hopefully successfully.

We visualize that there are two possible sets of tools, methods and procedures. The first set can already be existing and is being used presently as part of the present existing technologies, but the second set of tools and techniques would need more research work to attain them and to perfect them.

The first set of tools is what would be needed and used to inject/introduce the magnetic particles inside the eye. We visualize that we could use the rather conventional methods presently used to inject/insert/introduce these magnetic particles. We can use a rather small injection needle to do that. We will need to run enough experiments to determine the most effective size of the particles and then choose needles of appropriate size for the injection.

We may prefer to place the magnetic particles in a liquid of some sort to create an emulsion or slurry, and then inject this emulsion into the eye with the needle. We suspect that this procedure should almost be comparable to the present conventional method used to inject the Silicone Oil or the Air Bubbles in the eye.

Now the other side of the coin is what happens after the retina has been successfully and satisfactorily re-attached. We mean, how to extract the particles at the end of the process.

We could consider doing the following.

We can use the double-walled, telescoping, injection needles commonly used now for intravenous feeding or the like. The needle basically comprises two needles, one inside the other. The internal needle, or core needle, is similar to the usual metal needles, and it acts as the support and carrier of the external needle, or rather the external sheath. The external sheath is made out of a flexible material, like a plastic tube. It slides into the vein, or the artery, of the patient, together with, and with the support of, the internal metal core needle. Once the two needles are in the proper position, the internal needle, or core needle, is extracted, leaving behind the flexible sheath, to ultimately deliver the medications to the patient during any subsequent surgery or during the time the patient is staying in the hospital bed, say.

So, we could use a similar double-walled or telescoping needle for our present eye procedure. The outside sheath can be almost identical to the ones just described, but the inner core needle needs to be made of a magnetic material. We could insert this magnetic double-walled needle into the patient's eye, of course after the proper medical preparation, such as anesthesia, etc. Then we would pull the magnetic core needle out, extracting with it some of the magnetic particles from the inside of the eye to the outside, because some of the magnetic particles could have attached themselves to the magnetic core needle. We would wipe the magnetic particles off the magnetic core needle and after making sure that the core needle is properly cleaned, we would re-insert it back into the flexible sheath until it reaches the remaining magnetic particles, and then pull the core needle back out, again extracting with it some more magnetic particles. We can repeat this procedure until we are sure that we have extracted all the magnetic particles out from the eye.

This extraction procedure can be done while the surgeon or an assistant is watching the eye through proper optical instruments. Such an optical assessment could be good enough, to let the doctor decide whether all the magnetic particles have been removed from the eye.

It could also be possible to use some additional instrumentation to help in determining whether and when all the magnetic particles have been totally removed/extracted.

Proper research and experimentation will be needed to develop all the correct procedures and steps and about how and when to decide that all the magnetic particles have been extracted.

PS: the magnetic core needle can be a simple solid rod or wire, or it can be a hollow cannula, which could be used to “suck” out the magnetic particles.

Most of the above could be influenced by the size of the magnetic particles.

Which brings us to the SIZE OF THE MAGNETIC PARTICLES.

There are a few additional points to consider, especially regarding the size of the magnetic particles.

We guess that the smaller the better. We can even think about nano size particles. They could be easier to inject in the eye and probably to extract as well. However, we need to experiment to determine and to ascertain that such small particles would not be too small, so small that they could get lodged in certain areas of the eye anatomy, to create subsequent problems.

A counter consideration regarding the size of the particles is whether a larger size particle would work better with the magnets and the magnetic field and would provide a better or higher force to hold the retina against the eye walls.

Again, this needs to be determined, probably best empirically, by many actual experiments and evaluation of the results.

Another point to determine is whether the inside core needle should be a simple magnetic material, or whether ITS INTERNAL MAGNETIC ATTRACTION FORCE, i.e. the force inside the eye, could be augmented or enhanced by connecting its external end, the end which will be outside the eye, to a powerful magnet, be it a simple but strong permanent magnet, or be it connected to an electro magnet in some proper way. Or the whole eye and the extracting needle could be exposed to an external magnetic field, which could induce its field onto the particles and the needle and make them attract each other to close the magnetic field and thus get them to attach to each other.

The purpose of such a research effort would be to help in getting the magnetic particles out of the eyeball, all of it or at least as much of it as possible or as much as necessary.

The next phase of research is to conduct animal studies, to try to apply the proposed procedures on experimental animals, first dead ones, then live ones, one step or one phase at a time, and when the early stages or phases prove successful and satisfactory, then we can move on to the following ones. All this should be done BEFORE any of the proposed methods and techniques can be applied to human patients.

The ultimate goal would be to end up with a sort of an OPERATIONS MANUAL, covering the methods, materials, procedures, tools, specifications, etc. to help in teaching the practitioners of the proposed methods and to give them proper guidelines, so that they would do the best possible job and to accomplish and obtain the best possible results

One last issue to worry about is the following.

Having a metallic/magnetic material inside the patient's eye could create some problems, if and when the patient needs to undergo an MRI procedure or the like, or if he/she needs to go through security gates at an airport say. This may or may not be easy to solve, but it is something that we need to alert the patient about and that the patient needs to keep in mind.

1. A method for using magnetic material and a magnetic field for enhancing and promoting the healing of a repaired torn retina in the eye of a patient, the method comprising
the step of injecting magnetic material inside the eye of the patient, and
the step of placing at least one magnet adjacent to the patient's eye, and
positioning the magnet in a certain location so as to attract the magnetic material towards the area where the torn retina had been re-attached and to encourage the repaired retina to move closer to the walls of the eyeball.

2. A method as in claim 1, wherein
the magnetic material is bio-inert.

3. A method as in claim 1, wherein
the magnetic material is degradable.

4. A method as in claim 1, wherein
the magnet is placed outside the head of the patient,
carried by a certain carrying device, in such a way so as to enable the positioning of the magnet in any desirable position

5. A method as in claim 1, wherein
said magnet is an electro magnet, and wherein
said electro magnet can be turned on and off and can change the magnitude of the magnetic field, according the user's wishes and needs.

6. A method as in claim 3, wherein
at least one sensor is utilized to monitor the movement of the patient's eye, and wherein
said sensor will indirectly control the distribution and/or magnitude of the magnetic field, so as to influence the position of the magnetic material inside the eye.

7. A carrying device, to be carried by a patient, who had undergone a procedure, wherein certain magnetic material have been placed inside the patient's eye, wherein
the carrying device comprises
at least one magnet, which can be positioned such that its magnetic field would attract the magnetic material that are inside the eye to be urged to lean towards the area where the retina had been repaired and to urge that part of the retina to lean towards the adjacent walls of the eyeball.

8. A carrying device as in claim 7, wherein
said magnet is an electro magnet, and wherein
said electro magnet can be turned on and off and can change the conditions of the magnetic field, according the user's wishes and needs.

9. A carrying device as in claim 8, wherein
at least one sensor is utilized to monitor the movement of the patient's eye, wherein
said sensor will initiate a certain process to control the distribution and/or magnitude of the magnetic field, so as to optimize the conditions of the magnetic material inside the eye.

10. A set of cushions, comprising
at least one first cushion, which in turn comprises at least one compartment, which is reshapable under the influence of an external means and a control means, wherein
said cushion can be used as a support for part of the body of a user.

11. A set of cushions as in claim 10, wherein
said compartment of said first cushion is a sealed chamber, and
said external means is a source of compressed air, and wherein
the compressed air is applied to the sealed chamber of the cushion, and wherein
the user has access to a control means to adjust the internal pressure and shape and size of the cushion compartment, so that the cushion could properly support a certain part of the user's body.

12. A set of cushions as in claim 10, wherein
said set of cushions comprises a second cushion, and wherein
said second cushion is placed at a certain distance from said first cushion, such that the user can place his/her head between said first cushion and said second cushion, such that the patient's head can be maintained in a certain desirable position.

13. A set of cushions as in claim 12, wherein
at least one of said cushions can be positioned at a certain angle, to better maintain the position of the head of the patient at approximately a similar angle.

14. A set of cushions as in claim 12, wherein
at least one of said cushions comprises at least two segments, wherein
said two segments can be adjusted to have a certain angle between them, to cradle the patient's head.
